(19) United States
(12) Patent Application Publication
(10) Pub. No.: US 2010/0025263 A1
(43) Pub. Date: Feb. 4, 2010

(54) NANOPORE PARTICLE ANALYZER, METHOD OF PREPARATION AND USE THEREOF

(75) Inventors: Henry S. White, Salt Lake City, UT (US); Bo Zhang, State College, PA (US); Ryan J. White, Salt Lake City, UT (US); Eric N. Ervin, Salt Lake City, UT (US)

Correspondence Address: THORPE NORTH & WESTERN, LLP., P.O. Box 1219, SANDY, UT, 84091-1219, US

(73) Assignee: UNIVERSITY OF UTAH RESEARCH FOUNDATION, Salt Lake City, UT (US)

(21) Appl. No.: 11/743,536

(22) Filed: May 2, 2007

Related U.S. Application Data
(60) Provisional application No. 60/919,660, filed on Mar. 23, 2007.
(60) Provisional application No. 60/797,850, filed on May 5, 2006.

(51) Int. Cl. C12Q 1/06; G01N 27/28; G01N 27/26; B23P 11/00

(52) U.S. Cl. 205/777.5; 204/400; 205/775; 205/787; 205/789; 29/428; 977/904; 977/953

(57) ABSTRACT

Provided are the preparation, characterization, and application of a nanopore membrane device. The nanopore device comprises a thin membrane prepared from glass, fused silica, ceramics or quartz, containing one or more nanopores ranging from about 2 nm to about 500 nm. The nanopore is prepared by a template method using sharpened metal wires and the size of the pore opening can be controlled during fabrication by an electrical feedback circuit. The nanopore device is particularly useful for counting and analyzing nanoparticles of radius less than 400 nm.

### CROSS-REFERENCE TO RELATED APPLICATIONS

This application claims the benefit under 35 U.S.C. §119(e) of U.S. Provisional Application No. 60/919,660, filed Mar. 23, 2007 and US Provisional Application No. 60/797,850, filed May 5, 2006, the entirety of each of which is incorporated by this reference.

### STATEMENT REGARDING FEDERALLY SPONSORED RESEARCH OR DEVELOPMENT

This invention was made with government support under grant # FA9550 06-C-0060 awarded by the Defense Advance Research Projects Agency. This invention was also made with government support under grant CHE-0616505 awarded by the National Science Foundation. The US government may have certain rights to this invention.

### TECHNICAL FIELD

The invention relates to the field of nanotechnology. In particular, the invention is related to a glass nanopore device for counting and analyzing particles.

### BACKGROUND

Particle counting based on resistive pulse counting (or “electrozone sensing”) is a common method of particle analysis and is the basis of commercial Coulter Counters. In 1970s, DeBlois et al. reported the first use of a sub-Pn cylindrical pore etched in a plastic membrane in the detection of nanometer-sized particles (45 nm in radius) (DeBlois, R. W. and Bean, C. P. Rev. Scit. Instrum. 1970, 4, 909-916; DeBlois. R. W. and WYesley, R. K. A., J. Virol. 1977, 23, 227-233; and DeBlois, R. W. and Bean, C. P.; Wesley, R. K. A. J. Colloid Interface Sci. 1977, 61, 323-335). More recently, Crooks' group reported the applications of Si₃N₄ or PDMS supported epoxy membranes containing individual multi-walled carbon nanotube (˜65 nm in radius); particles with different size and surface charge were simultaneously analyzed (Sun. L. and Crooks, R. M. J. Am. Chem. Soc. 2000, 122, 12340-12345; Ito, T., Sun, L. and Crooks, R. M. Anal Chem. 2003, 75, 2399-2406; Ito, T., Sun, L. Bevan and M. A.; Crooks, R. M. Langmuir. 2004, 20, 6940-6945; Ito. T., Sun L. and Crooks, R. M. Chem. Comm. 2003, 1482-1483; Henriquez, R. R., Ito, T., Sun, L. and Crooks, R. M. Analyst. 2004, 129, 478-482; and Ito, T., Sun, L., Henriquez, R. R. and Crooks, R. M. Acc. Chem. Res. 20, 937-945). Sohn's group showed the successful application of micro-fabricated nanopores/channels in quartz substrate/PDMS membranes in counting of nanoparticles (as small as 43 nm in radius, ˜0.16 pM) and biological molecules, and in the sensing of biological interactions (Saleh, O. A. and Sohn, L. L. Rev. Sci. Instrum. 2001, 72, 4449-4451; Saleh, O. A. and Sohn, L. L. Proc. Nati. Acad. Sci. U.S.A. 2003, 100, 820-824; Saleh, O. A. and Sohn, L. L. Rev. Sci. Instrum. 2002, 73, 4396-4398; Salch, O. A. and Sohn, L. L. Nano Lett. 2003, 3, 37-38). Other techniques, such as dynamic light scattering (Russel, W. B., Saville, D. A. and Schowalter, W. R. Colloidal Dispersions, Cambridge University Press, New York, 1989) and field-flow fractionation (FFF), (Giddings, J. C. Unified Separation Science. John Wiley & Sons, Inc. 1991) have been successfully applied in the analysis of nanoparticles. Single protein ion-channels (e.g., α-hemolysin) have also been utilized as sensing elements for single molecule detection (Bezrukov, S. M. and Kasianowicz, J. J. Eur. Biophys. J. 1997, 26, 471-476; Kasianowicz, J. J., Brandin, E., Branton, D. and Deamer, D. W. Proc. Natl. Acad Sci. USA 196, 93, 13770-13773; Meller, A., Nivon, L., Brandin, E., Golovchenko, J. and Branton, D. Proc. Natl. Acad. Sci. USA 2000, 97, 1079-1084; Deamer, D. W. and Branton, D. Acc. Chem. Res. 2002, 35, 817-825; Bayley, H. and Cremer, P. S. Nature 2001, 413, 226-230; Howorka, S., Cheley, S. and Bayley, H. Nature Biotech. 2001, 19, 636-639).

Commercial instruments (e.g., MULTISIZER™ 3 COULTER COUNTER®, Beckman Coulter, Inc.) allow for detection of particles no smaller than 200 nm in radii. However, applications of smaller nanoparticles (e.g., less than 100 nm) in fundamental and applied research areas require new analytical techniques that allow easy and accurate detection of particle size and concentration.

### SUMMARY OF INVENTION

Provided is a nanopore device, the device comprising: a membrane having a thickness, having a first and second side, the first side being opposite to the second sides and having a nanopore extending through the membrane over the thickness of the membrane. Typically, the membrane containing a nanopore separates two compartments, which two compartments typically contain electrolyte solutions. The device may further comprise a means for applying an electric field between the first side and the second side of the membrane; a means for monitoring the current flow through the nanopore or resistance between the first side and the second side of the membrane, and a means for processing the observed current or resistance to produce a useful output. Various embodiments of the nanopore device may be incorporated into larger device structures that provide supporting elements for, for example, data acquisition and analysis.

In certain embodiments, the membrane may be made of glass, Si, SiO₂, Si₃N₄, quartz, alumina, nitrides, metals, polymers or other suitable materials. The membrane can be of a pure substance or a composite, or if necessary, comprises a coating that modifies the surface of the material. The thickness of the membrane is typically the smallest dimension of the membrane. The membrane ranges typically from about 10 μm to several hundreds of micrometer in thickness.

The device may further comprise a chamber wherein the membrane is an integral part, such as, for example, of the bottom or the side walls, of the chamber. In a particular embodiment, a single nanopore is fabricated in a thin glass membrane located at the bottom side of a glass capillary.

The membrane may be configured to include more than one nanopore, or an array of nanopores. Each individual nanopore may be enclosed in an individual chamber and such individual chambers may be arranged in an array format on suitable support structures.

In various embodiments, the nanopore has a first opening and a second opening. The first opening opens to the first side of the membrane and the second opening opens to the second side of the membrane. The two openings may be of different sizes or shapes. Preferably, the first opening is smaller than the second opening. In particular, the nanopore is of an about truncated conical shape, wherein the first opening is smaller the second opening. The radius of the first opening of the nanopore preferably ranges from about 2 nm to about 500 nm, or larger. The radius of the second opening can be about 5 μm to 25 μm. Since the nanopore extends through the membrane, and connects the first side and the second side of the membrane, the thickness of the membrane is typically the length or depth of the nanopore if the thickness of the membrane is uniform across the membrane. The length of the nanopore is preferably 20 times of the radius of the first opening of the nanopore. The length of the nanopore may range from about 20 μm to about 75 μm. The position of the nanopore may be located at any predetermined position on the membrane.

The “means for applying an electrical field” typically comprises a first electrode positioned on the first side of the membrane, and a second electrode positioned on the second side of the membrane. The first and second electrodes may be made of any suitable material(s), such as, for example, Ag/AgCl. The first and second electrodes are usually positioned on opposite sides of the membrane. However, it is to be understood that positioning of the first and second electrodes is relative in relation to the first and the second sides of the membrane. For example, if the second side of the membrane is enclosed in a chamber, and the first side of the membrane is outside that chamber, then, the first electrode is positioned outside the chamber, while the second electrode is positioned inside the chamber.

Further provided herein is a method of forming a nanopore device, the method comprising: providing a membrane having a thickness, having a first side and a second side, and having a nanopore extending through the membrane over the thickness of the membrane; providing a first electrode being positioned on the first side of the membrane and a second electrode being positioned on the second side of the membrane; providing a means for monitoring the current flow through the nanopore or resistance between the first side and the second side of the membrane; and providing a processing means that process the observed current and resistance to produce a useful output.

In certain embodiments, the invention provides a nanopore particle analyzer. The nanopore particle analyzer comprises a chamber wherein a membrane is an integral part of the chamber, a nanopore extending trough the membrane over the thickness of the membrane, a first electrode being positioned outside the chamber, a second electrode being positioned inside the chamber, a means that applies electrical field between the first and the second electrode, a means for monitoring the current flow through the nanopore or resistance between the first side and the second side of the membrane, and a processing means that process the observed current ad resistance to produce a useful output. In particular, the chamber may be a glass chamber comprising the glass membrane as the bottom wall of the chamber.

The nanopore has a first opening and a second opening. Preferably, the nanopore is of a conical shape, with the first opening smaller than the second opening. The first opening is facing outside of the chamber and the second opening is facing inside of the chamber. The first opening of the nanopore preferably ranges from about 2 nm to about 500 nm. The chamber may contain an appropriate electrolyte solution, e.g., KCl, NaCl, phosphate buffered saline (“PBS”), any other suitable salt solution, wherein the second opening is submerged in the electrolyte solution and the appropriate part of the second electrode is immersed in the electrolyte solution.

Further provided herein is a method of counting and analyzing particles using the nanopore particle analyzer as disclosed herein, the method comprising: providing a sample containing particles to be analyzed, contacting the nanopore particle analyzer such that the first opening of the nanopore is immersed in the sample, and the appropriate part of the first electrode is immersed in the sample; applying an appropriate voltage between the first and the second electrode of the nanopore analyzer such that the particles from the sample solution are driven to pass across the nanopore; monitoring the transient change in the electrical resistance, or electrical conductivity of the nanopore; and analyzing the transient change to obtain the concentration, size, shape and/or electrical charge of the particles. DC or AC voltage may be applied via the electrical field applying means. Typical DC voltage ranges from about 10 to about 500 mV. Typical AC voltage ranges from about 2 to about 25 mV rms. This method can be used to analyze various particles, including but not limited to cells, bacteria, viruses, polymeric particles, ions and molecules. The particle analyzer allows measurement of particles from about 2 nm to about 500 nm.

### DESCRIPTION OF THE FIGURES

FIG. 1 is a cut away, side schematic of a conical shaped nanopore in a thin glass membrane.

FIGS. 2A and 2B schematically depict a nanopore particle analyzer.

FIG. 3 depicts (A) Voltammetric response of a 62-nm-radius Pt disk electrode in H₂O containing 10 mM Ru(NH₃)₆Cl₃ and 0.1 M KCl, and (B) the i-V response of the corresponding nanopore membrane (Pt removed) in 0.5 M KCl and in 0.1 M KCl.

FIG. 4 shows detection of 45-nm radius negatively charged polystyrene particles. FIG. 4(A) shows current-time recording of a 62-nm-radius glass nanopore in 0.1 M KCl with 10 mM PBS buffer (pH 7.4) at Vₒₚₚ=−0.3 V; FIG. 4(B) shows current-time recording of the same glass nanopore as in (A) in the presence of 2.4×10⁹/ml particles at Vₐₚₚ=−0.3 V; and FIG. 4 (C) Current-time recording of the same glass nanopore in the presence of 2.4×10⁹/ml particles at Vₐₚₚ=+0.3 V.

FIG. 5 shows current-time recording of the 62-nm-radius glass nanopore in 0.1 M KCl with 10 mM PBS buffer (at pH=7.4) in the presence of 45-nm radius panicles at different concentrations: (A) 2.4×10¹¹/ml, (B) 2.4×10¹⁰/ml, (C) 2.4×10⁹/ml, and (D) 2.4×10⁸/ml. FIG. 5 (E) shows the log plot of rate as a function of particle concentration.

FIG. 6 is a graph showing the rate of 45-nm radius particle transfer as a function of applied voltage.

FIG. 7 are graphs showing detection of 30-nm radius positively charged polystyrene particles. FIG. 7(A) shows an i-V recording of the 64-nm-radius glass nanopore in 0.5 M KCl with 10 mM PBS buffer (pH=7.4) at Vₐₚₚ=0.2 V; and FIG. 7(B) shows a current-time recording of the same glass nanopore as in FIG. 7(A) in the presence of 8×10¹¹/ml particles at Vₐₚₚ=0.3 V.

FIG. 8(A) shows an i-t recording of a 64 nm radius glass nanopore in 0.5 M KCl with 10 mM PBS buffer (ph=7.4) in the presence of 8×10¹¹/ml particles. A voltage of −0.3 is applied at the beginning, it is then changed to +0.3 V for ˜2 seconds and then changed back to −0.3 V. FIGS. 8(B), (C), and (D) are the same plot as in (A) but show only the initial part (B), the middle (D) and the last part (C).

FIG. 9 (A) shows a typical current pulse from FIG. 8(C) corresponding to a particle translocates from the bulk solution into the glass capillary and a cartoon showing the direction of the particle movement. FIG. 9(B) shows a typical current pulse from FIG. 8(D) corresponding to a particle translocates from the glass capillary through the glass nanopore into the bulk solution and a cartoon showing the direction of particle movement.

FIG. 10 shows the geometry of a nanopore membrane and an electrochemical cell used in the simulation (not drawn to scale).

FIG. 11 is a schematic drawing of the relative size of a glass nanopore membrane and a nanoparticle in the pore mouth. The dotted circle shows the area that the nanoparticle can transfer through the pre, which has a radius of rₗ-rₚ.

FIG. 12 is the simulated distribution of electrical field in the electrochemical cell in the absence of nanoparticles at Vₐₚₚ=3 mV.

FIG. 13 is a graph showing the computed particle transfer rate as a function of applied voltage and particle charge.

FIG. 14(A) shows a simulated current pulse and FIG. 14(B) shows a typical current pulse recorded in the experiment of FIG. 5.

FIG. 15 are graphs showing that the detection of nanoparticles obeys Poisson distribution: (A) showing transport of positively charged 30-nm radius particles with 10-ms counting interval, and (B) showing transport of negatively charged 45-n radius panicle with 100-ms counting interval.

### DETAILED DESCRIPTION OF THE INVENTION

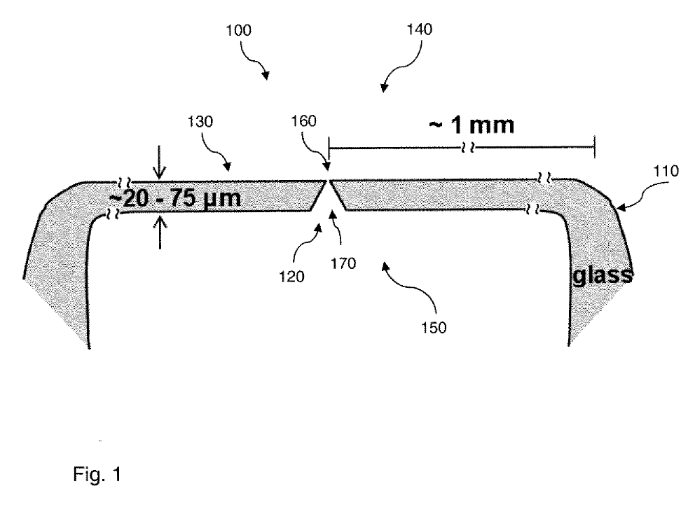

FIG. 1 is a cross-sectional view of a conical shaped nanopore in a thin glass membrane. In FIG. 1, nanopore device, generally 100, comprises glass capillary 110, and nanopore 120. Glass membrane 130 is an integral part of glass capillary 110. Glass membrane 130 has a first side 140 and a second side 150. Nanopore 120 extends through glass membrane 130, thus forms a channel connecting the first side and the second side of glass membrane 130. Nanopore 120 has first opening 160 facing the first side of glass membrane 130, and second opening 170 facing the second side of glass membrane 130. First opening 160 is smaller than second opening 170. Typically, first opening 160 is ranging from 2 nm to 500 nm, and second opening is ranging from 5 μm to 25 μm. The thickness of glass membrane 130, also the length of nanopore 120 in this case, is ˜20-75 μm.

Although a nanopore can be made of various suitable shapes, a conical shaped nanopore is preferred. Two advantages are associated with the conical shape pores. First, higher ionic conductivity can be achieved with conical shaped pores relative to cylindrical pores without sacrificing the ability to localize the resistance to the pore orifice (Li, N; Yu, S.; Harrell, C. CA; Main, C. R. Anal. Chem. 2004, 76, 2025). Second, the steady-state flux of molecules (or ionic conductivity) in a conical shaped pore is independent of the pore depth for pores that have a length >20 times of the orifice radii of the smaller opening (Zhang, B. Zhang, Y. and White, H. S. Anal. Chem. 2004, 76, 6229; Zhang, B., Zhang, Y. and White, H. S. Anal. Chem. 2006, 78, 477; Zhang, Y. Zhang, B. and White, H. S. J. Phys. Chem. B 2006, 110, 1768). This characteristic is potentially very important in the fabrication of nanopores that exhibit reproducible behavior.

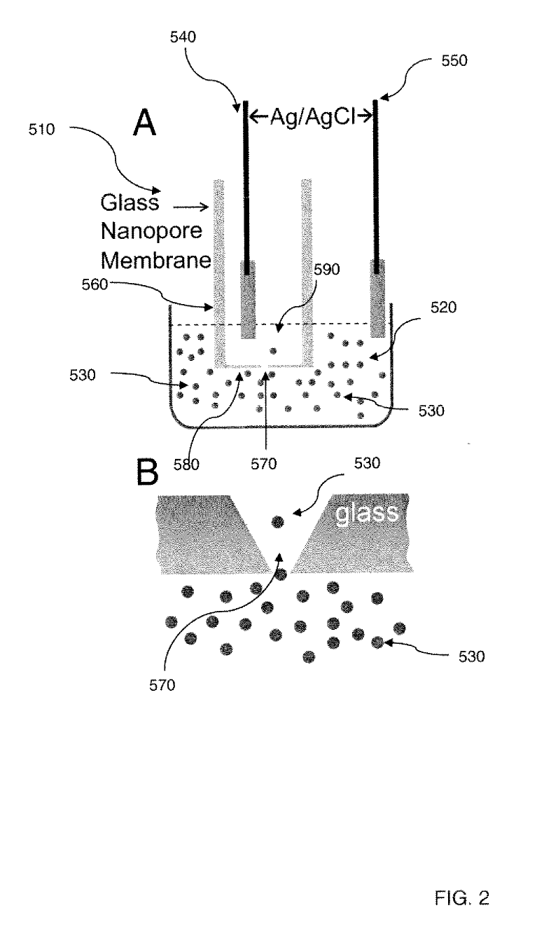

FIG. 2 is a schematic of a nanopore particle analyzer. Glass nanopore device 510 comprises glass chamber 560, electrode 540 and electrode 550. Glass membrane 580 is an integral part of glass chamber 560. Nanopore 570 is included in glass membrane 580. Chamber 560 contains electrolyte solution 590. Device 510 is placed in sample 520, which contains particle analytes 530. Nanopore 570 is of a conical shape, with the smaller opening of the nanopore contacting sample 520. The smaller opening of nanopore 570 ranges from 2 nm to 500 nm. Electrode 540 is positioned inside glass chamber 560 and the appropriate part of electrode 540 is immersed in solution 590. Electrode 550 is placed in sample 520 and the appropriate part of the electrode 550 is immersed in sample 520. A voltage is applied between electrodes 540 and 550 to drive an ionic current through nanopore 570. Particles passing through nanopore 570 are readily detected by measuring the transient change in the electrical resistance, or electrical conductivity of nanopore 570. As particles pass through the nanopore, a short transient decrease in the current is observed. The frequency of these resistive pulses is proportional to the particle concentration, while the magnitude and shape of the pulse provides the nanoparticle shape and size. The shape and duration of the pulse can be used to determine the shape, size, and/or charge of a particle. Frequency of pulses may also indicate the concentration of a particle. This method can be used to determine the concentration, the shape, the size and electrical charge of the particles.

The nanopore particle analyzer is ideal for analysis of panicles in the 5-100 nm range, but may be used for measurement of particles smaller than 5 nm or bigger than 100 nm. Various particles, including but not limited to cells, bacteria, viruses, polymeric particles, ions, molecules, and nanoparticles that are used for formulating and delivery of small molecule, peptide or macromolecular drugs. The nanopore particle analyzer can also be used in environmental water analysis and as sensors in homeland security and military applications. Exploitation of the present invention will be driven by the explosive growth of new technologies based on nanoparticles and by new regulations in environmental monitoring.

The invention is further described with the aid of the following illustrative Examples.

Fabrication of a Nanopore membrane A nanopore membrane may be prepared by the following procedures: (1) a template, preferably a signal transduction element, with an atomically sharp tip is prepared; (2) the tip of the template is sealed a substrate; (3) the substrate is polished in order to expose the tip of the template; (4) the exposed part of the template is etched to produce a nanopore in a substrate; and (5) the template is removed from the substrate to leave a nanopore in the substrate. Some fabrication methods of glass nanopores are disclosed in Zhang, Anal Chem. 2004, Zhang, Anal. Chem., 2006; Zhang, J P C, 2006, Wang, JACS 2006, R. J. White et al., Langmuir 22, 10777 (2006). The following provides an example of fabrication of a glass nanopore membrane.

A 1-cm length piece of 25-km-diameter Pt wire (Alfa-Aesar, 99.95%) is electrically contacted to a W rod using Ag conductive epoxy (DuPont). The end of metal wire is electrochemically etched to an atomically sharp point, and a 20-70 μm part of the tip is then sealed into soda-lime glass capillary (Dagan Corp., SB16, 1.65-mm o. d., 0.75-mm i. d., softening point=700° C.) using H₂ flame: the glass capillary is melted using the middle part of the flame with the Pt tip ˜10 mm away from the end. The tip is then inserted to approach the melted end without physical touching. The glass is then heated again using the lower part of the flame. A bright flat surface could be found in the melted part of the glass capillary, which is then used to determine the sealing of Pt tip. The insertion of the Pt tip into the flat glass surface could be easily noticed as the appearance of a small spot. The electrode is then imrmediately moved out of the flame and cooled down at room temperature. The electrode is then polished until the exposure of a nanometer-sized Pt disk. In order to make a glass nanopore, the Pt is electrochemically etched in a CaCl₂ solution using an AC voltage (˜3 V).

The geometry of a conical shape glass nanopore can be fully described using any three of four parameters: the radius of the small opening, a; the radius of the large opening, r, the half-cone angel, θ: and the length of the pore, L.

The size of the small pore opening can be determined by two methods. It can be measured by the steady-state limiting current of a redox species before the Pt is etched away, using the following equation:

i_{d}=4nFDC_{b}a. (1)

where, n is the number of electrons transferred per molecule, F is Faraday's constant, and D and C_{b} are the diffusion coefficient and bulk concentration of the redox molecule, respectively. It can also be calculated from the electrical resistance of the conical shaped pore, R, assuming unchanged geometry upon removing Pt, using the following equation:

$$R = \frac{1}{κ a} ( \frac{1}{4} + \frac{1}{π tan ( θ )} ) \qquad ( 2 )$$

where, κ is the conductivity of the KCl solution (˜5.5 S/m for 0.5 M KCG). The angle θ can be determined using an optical microscope and is usually between 7 and 12° when etched in NaCN.

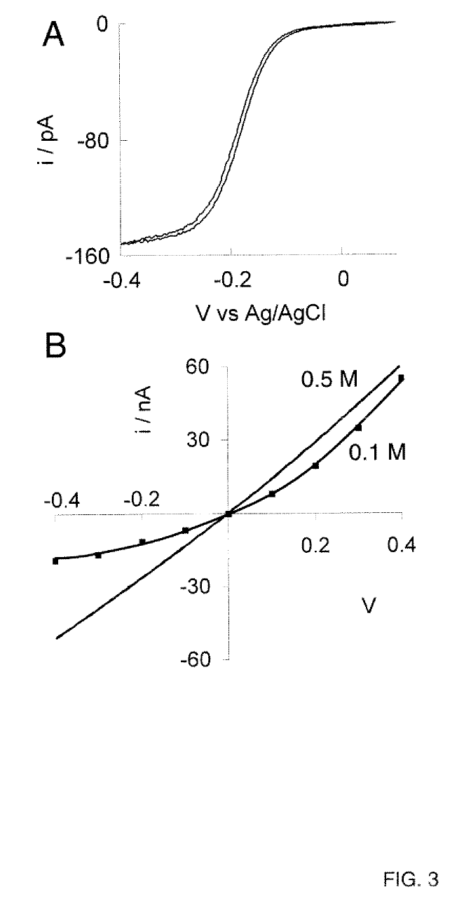

As an example, FIG. 3(A) shows the voltammetric response of a 62-nm-radius Pt disk electrode in 10 mM Ru(NH₃)₆Cl₃ containing 0.1 M KCl. The radius of the Pt is calculated from the steady-state limiting current, using equation 1. FIG. 3(B) shows the i-v response of the glass nanopore membrane made from the same electrode, in KCl solutions containing 10 mM buffer (pH=7.4) and 0.1% of triton X-100. The i-V response is linear in 0.5 M KCl, whereas it shows nonlinearity in the solution containing 0.1 M KCl. The current rectifying effect is believed to be because of the asymmetry of the conical-shape pore and surface charge on glass walls. The D.C. resistance is measured to be ˜7.5 MΩ in 0.5 M KCl, which yields a pore radius to be 61 nm based on the measured half-cone angle of ˜8° in good agreement with the value by electrochemical measurements using equation 1.

FIG. 2 shows the experimental setup for detecting nanoparticles using glass nanopore membranes: a glass capillary containing an individual cone-shape pore is placed in a cell containing 0.1 M KC buffered with 10 mMv PBS at a pH=7.4. The same solution is injected to the same level into the glass capillary using a home-made micropipette to avoid hydrostatic pressure gradients. Two Ag/AgCl electrodes are placed in each solution to drive the current across the membrane.

A CHEM-CLAMP (CORNERSTONE Series) Voltammeter-Amperometer or other appropriate electrical instrument is used to apply the voltage difference between inside and outside the glass capillary and to measure the resulting current. Data were digitized using a National Instruments PCI-6251 Multifunction I/O & Ni-DAQ card (National Instruments) and recorded using in-house virtual instrumentation written in LabVIEW 6.0 (National Instruments) at a sampling frequency of 100 kHz. A 3-pole Bessel low-pass filter was applied at a cut-off frequency of 10 KHz. Voltages are defined between the electrode outside the capillary vs. the electrode inside.

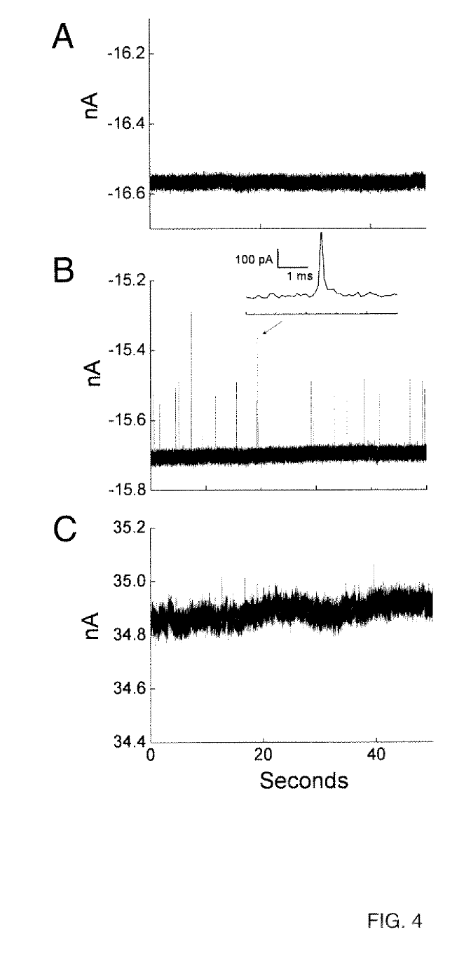

As an example, the above glass nanopore membrane is used to detect negatively charged 45-nm-radius polystyrene (PS) particles (with ˜42,000-COOH groups). FIG. 4(A) shows the i-t trace of the glass nanopore at Vₐₚₚ=300 mV in 0.1 M KCl solution buffered at pH=7.4 containing 0.1% Triton X-100 before adding polystyrene particles. A constant current (˜16.6 nA) is observed. FIG. 4(B) shows the current-time response of the same glass nanopore in the same KCl solution in the presence of PS particles (2.4×10⁹/ml). Current pulses are observed, corresponding to translocation of individual nanoparticles through the glass nanopore. A typical enlarged current pulse is shown as the inset. As a control experiment, FIG. 4(C) shows the i-t recording when a positive voltage is applied (Vₐₚₚ=+300 mV other experimental conditions the same as in 3b). No signals are observed, since the negatively charged particles are repelled away from the pore orifice. The current magnitude (˜34.8 nA) is much larger than that in FIG. 4(B) because of the rectification effect of the asymmetric nanopore. FIG. 3(B).

Unlike the typical square-ware current pulses obtained using cylindrical pores, the current pulses using the glass nanopores have a quasi-triangle wave shape, which is due to the conical shape of the glass nanopores. As reported previously (Zhang, B.; Zhang, Y.; White. H. S. Anal. Chem. 2004, 76, 6229-6238. Zhang, B.; Zhang, Y., White, H. S. Anal. Chem. 2006, 78, 477-483), the mass-transfer resistance inside a conical-shaped nanopore is localized at the small pore-orifice. Thus, the resistance change (increase) is largest when a nanoparticle is in immediate vicinity of the pore orifice. A maximum decrease in current is anticipated when the particle passes through the pore orifice. In contrast, the change in the resistance of a cylindrical pore will be approximately constant as a particle travels the length of a pore (DeBlois, R. W.; Bean, C. P. Rev. Sci. Instrum. 1970, 41, 909-916). Thus, the decrease in current remains constant as the particle translocates, which corresponds a square wave pulse in the i-t response.

The average pulse width in the conical shaped pore is ˜80 μs in this condition (300 mV bias voltage, 45 nm radius particle), which is 1-2 orders of magnitude smaller than pulse widths measured using cylindrical nanopore systems for similar conditions. This greatly enhances the resolution of pulse signals and thus may provide a lower detection limit. There are two reasons which might contribute to the shorter pulse width. First, when using a conical pore, the length of “sensing zone” is greatly shortened as described above. In other words, the “sensing zone” is also localized at the small orifice (instead of spanning the entire length of pore for cylindrical geometry). Second, the velocity of particle traveling through the “sensing zone” is likely to be higher for a conical pore than for a cylindrical pore of the same diameter and same length. Numerical simulations show that the voltage drop across the nanopore membrane is localized near the pore orifice for a conical pore (in the “sensing zone”), where the electric field is much higher than any other regions inside the pore. For a cylindrical pore, the same voltage drop is distributed in a much wider “sensing zone”. Thus, the electric field is also smaller than for a conical pore. The electrophoretic velocity is proportional to the electrical field,

qE=f′V (3)

where, q is the charges on a single particle, E is the local electric field, f′ is the friction coefficient of a single particle, which is a fundamental parameter reflecting the magnitude of drag forces through fluids and can be given by Nernst-Einstein equation

$$( D = \frac{RT}{f} ) ,$$

and V is the velocity of the particle. The electrophoretic velocity is higher in a conical pore than in a cylindrical pore (of the same diameter and length).

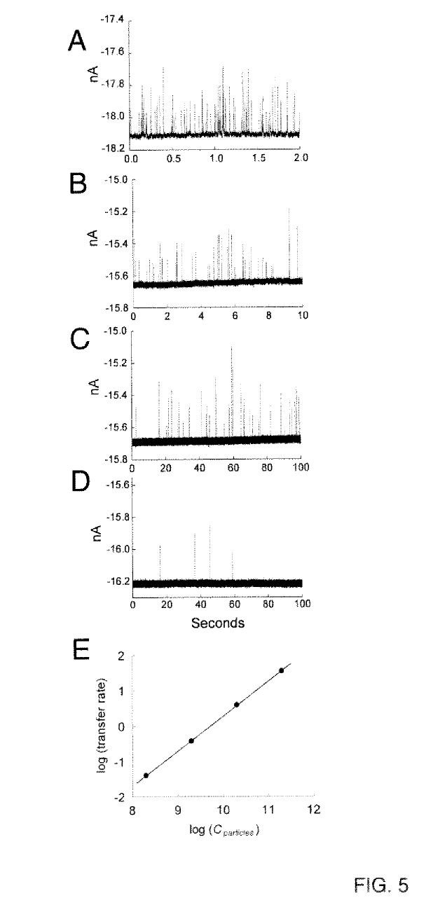

A linear dependence is found between the translocation rate and the particle concentration. FIG. 5(A) through 5(D) show i-t recordings of the 62-nm-radius glass nanopore membrane in 0.1 M KCl and 10 mM PBS buffered at pH=7.4, containing different concentrations of 45-nm-radius negatively charged PS particles. FIG. 5(E) shows a log plot of the translocation rate as a function of particle concentration. The slope is 0.99, indicating good linear dependence between counting rate and the particle concentration. Particles with concentrations as low as 0.41 pM have been detected in ˜10 minutes (˜22 counts detected). Lower particle concentrations can be detected by this method.

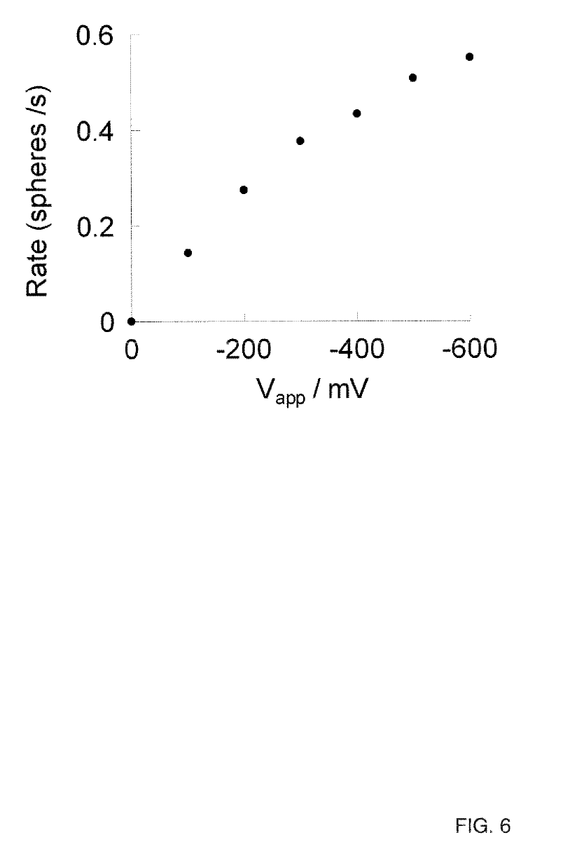

FIG. 6 shows the translocation rate as a function of the applied voltage for the counting of negatively charged 45-nm-radius PS nanoparticles using a 62-nm-radius glass nanopore. The obtained translocation rate is proportional to the applied voltage when it is less than ˜200 mV, and then levels off when higher voltages are applied. As is shown later in the simulation, the translocation rate should be proportional to the applied voltage. The reason for the discrepancy is believed to be from the surface charges and the asymmetry of the glass nanopore. As shown in FIG. 3(B), the i-V response is rectified (non-linear). When a positive voltage is applied from the big pore opening to the small opening (same condition as in the detection experiment shown in FIG. 6), the current levels off as a result of redistribution of counter ions in the electrical double layer in the pore. Because the ionic current is proportional to the flux of the ionic species through the pore, the flux of ionic species is also rectified.

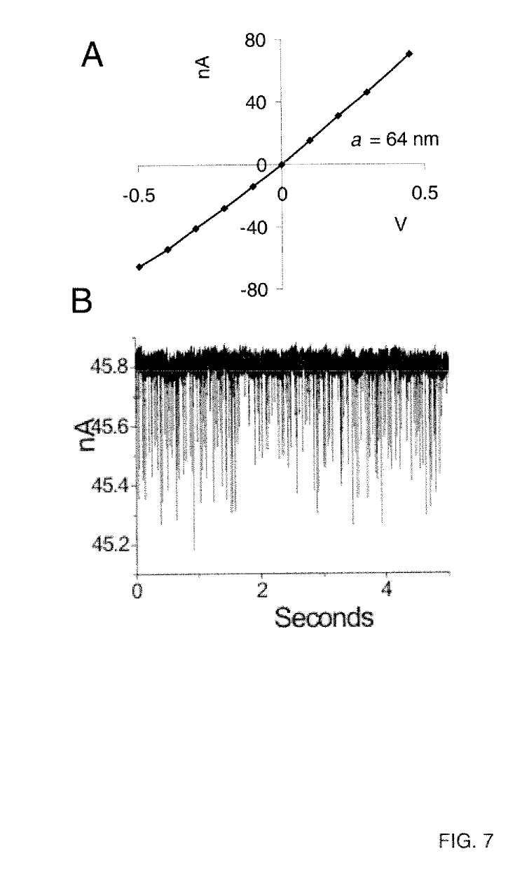

Detection of 30-nm-radius Polystyrene Nanoparticles. 30-nm-radius positively charged PS particles are detected using a 64-nm-radius glass nanopore membrane. FIG. 7(A) shows the i-v response of the glass nanopore membrane, in 0.5 M KCl solution containing 10 mM buffer (pH=7.4) and 0.1% of triton X-100. The DC resistance yields a pore radius to be 64 nm. FIG. 3(B) shows the i-t recording of the glass nanopore membrane at +300 mV in 0.5 M KCl buffered at pH=7.4 containing 0.1% Triton X-100 in the presence of 30-nm-radius positively charged PS particles (8×10¹¹/ml).

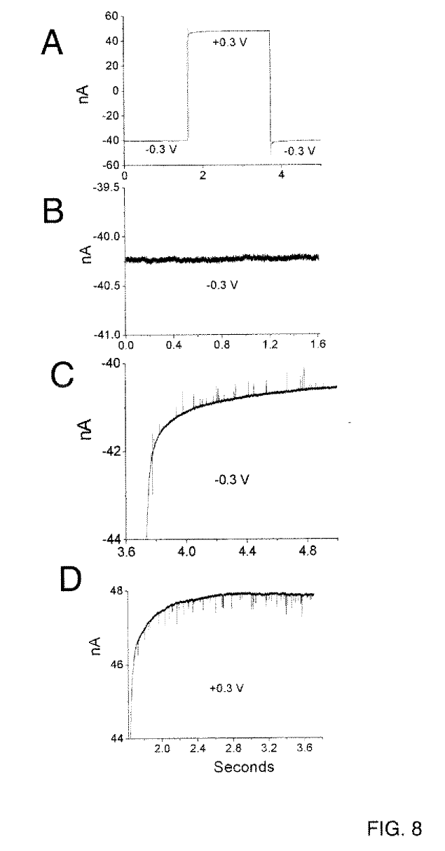

The applied voltage is switched from −300 mV to +300 mV then to −300 mV to observe the dependence of the current pulse-shape on the direction of particle translocation. As shown in FIG. 8, at the beginning, at −3 mV, positively charged particles are attracted from the pore orifice. No resistive pulses are observed. When +300 mV is applied, downward current pulses are observed corresponding to the particles electrophoretically driven into the pore. When 300 mV is applied immediately after the +300 mV, upward current pulses are observed corresponding to the particles electrophoretically driven from inside the glass capillary back into the bulk solution.

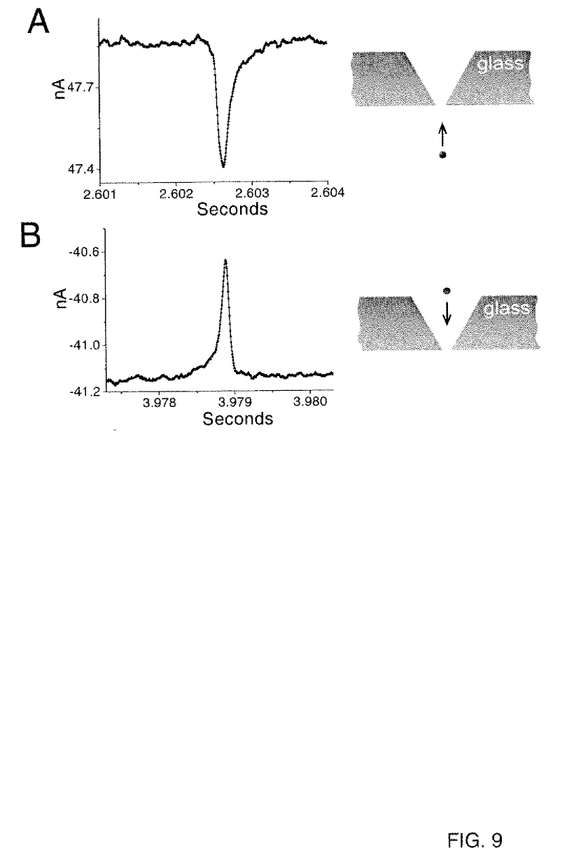

FIG. 9 shows two typical current pulses from FIG. 8. FIG. 9(A) shows a current pulse corresponding to the translocation of nanoparticles from bulk solution into the glass capillary. The current decrease is sharper when a particle moves from the bulk solution to the pore orifice, whereas it increases slowly to the baseline current when it moves from the pore orifice to the glass capillary. FIG. 9(B) shows a current pulse corresponding to nanoparticles electrophoretically driven back into bulk solution. The current first slowly decreases to a minimum value corresponding to the particle being electrophoretically driven from the glass capillary to the pore orifice. The current rapidly increases to the base line current corresponding to the particle being driven away from the orifice to the bulk solution. The absolute values of base line current are different due to the rectification effect of the pore walls. The pulse shapes of the two current traces look otherwise quite similar to each other (inversely placed). The results indicate the shape of the current pulse indeed reflects the mass transfer resistance as a function of the position inside/near to the conical shape pore.

Finite-Element Simulations of Nanoparticle Detection using Glass Nanopore. For comparison to experiment, the rate of particle detection and the shape of the current pulse are simulated using finite-element simulation. The finite element simulations provide validation of the experimental results using the nanopore membrane, specifically demonstrating that the measured translocation times and counting rates are in agreement with well-known physical theory.

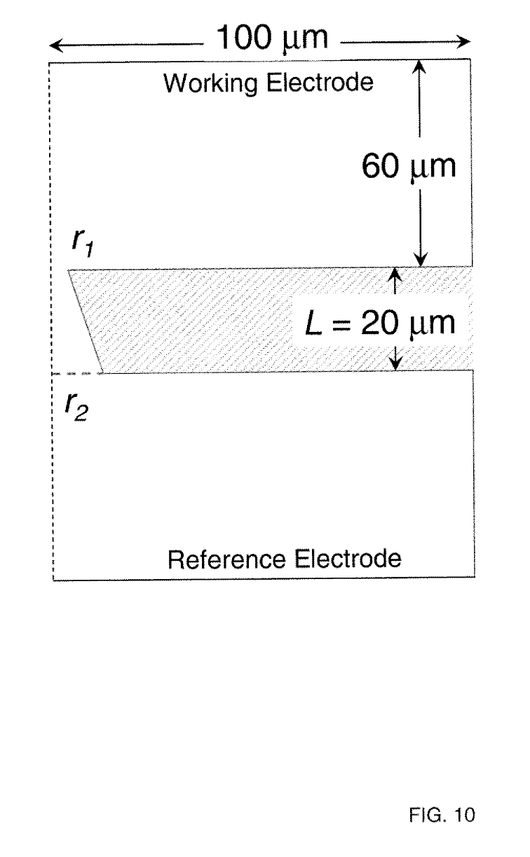

The geometry of the electrochemical cell and the glass nanopore membrane is shown in FIG. 10. The nanopore membrane is simulated using a cylindrical coordinate system with axial symmetry. The origin (z=0, r=0) corresponds to center of the small orifice. The glass membrane is the shaded area in FIG. 10 with a thickness of 20 μm. This value is large enough for a conical nanopore to display constant resistance (˜320× larger than the radius of the pore orifice). To approximate the semi-infinite boundary condition of the experiment, the boundaries are set 60 μm in the z direction away from the glass membrane surface and 100 μm in the r direction away from the center of conical pore.

The boundaries shown in red lines are set as insulating boundaries (flux=0). The black dashed line is an axial symmetry boundary. The green dashed line is an interior boundary for integrating total flux of the particles through the pore. One electrode is placed outside the glass capillary (facing the small pore opening), while the second electrode is placed inside the glass capillary, facing the large pore opening. The model does not consider the surface charges on pore walls. Thus, the effect of electrical double layer is not considered in the simulation.

The flux equation used in the simulation is the Nernst-Planck equation. For simplicity, only K⁺, Cl⁻, and PS spheres are assumed in the system. The diffusion coefficient of K⁺ and Cl⁻ are set to be 1.8×10⁻⁹ m²/s and 2.0×10⁻⁹ m²/s, respectively. The diffusion coefficients for 45-nm-radius and 30-nm-radius spheres are calculated to be 4.5×10⁻¹² m²/s and 7.33×10⁻¹² m²/s, respectively, based on the Stokes' law. The number of negative surface charges (˜1500) on the 45-nm-radius particle is estimated using the number total surface functional groups and the fractional number (˜3-4%) of COOH that are deprotonated. The number of positive surface charges (˜50) on the 30-nm-radius particle is estimated by a finite-element simulation of the transfer flux as a function of the applied voltage.

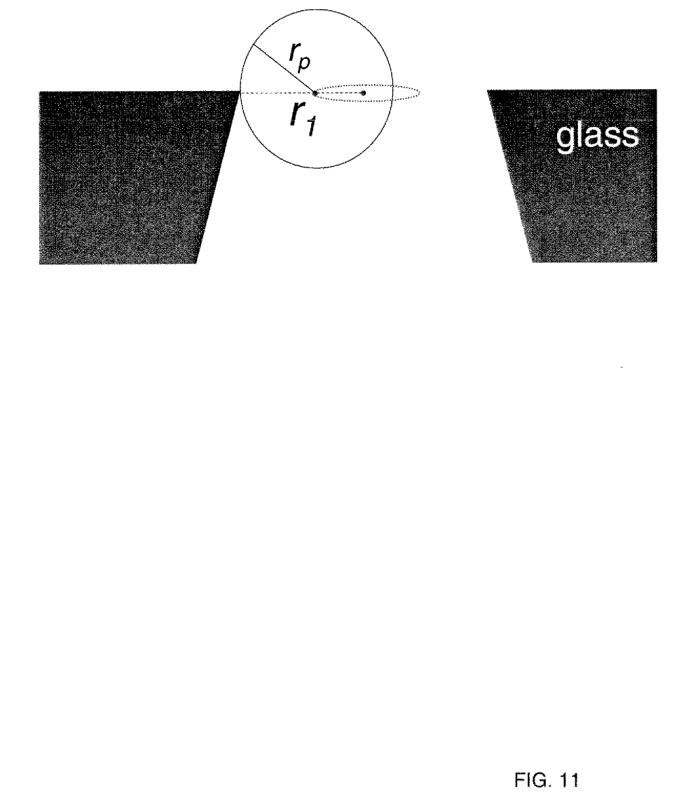

In computing the particle flux through the pore, the particles are treated as point charges. However, as shown in FIG. 11 since the particles have a finite radius, only those particles within a distance rₗ-rₚ of the pore center can translocate through the pore. Thus, for the experiments described in FIG. 4, the effective radius of pore in the simulation is set to be 17 nm.

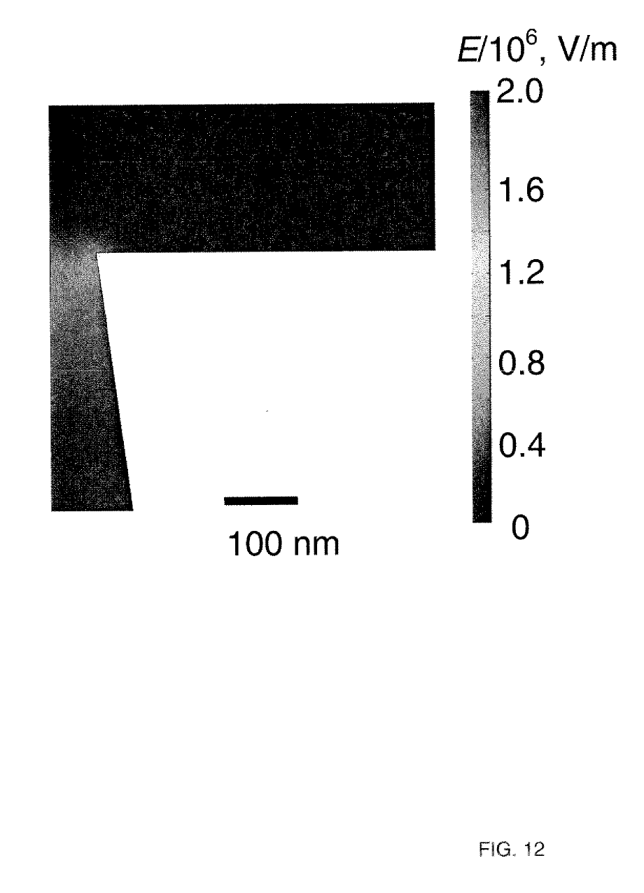

In a separate simulation, the determination of current pulse shape is performed by manually moving a sphere (30-nm-radius) along the center of the pore, in small steps (step size=50 nm and 100 nm, depending on the distance of the particle away from the pore orifice), beginning from ˜10 μm away from the pore. The concentration of KCl (0.5 M) and the applied voltage are held constant (300 mV) throughout the simulation. At each position, the current is simulated in the presence of the particle. FIG. 12 shows a simulated distribution of die electrical field in the electrochemical cell. The electrical field at the nanoparticle surface is then used to compute the electrophoretic velocity, using equation 3.

The calculated electrophoretic velocity is then used to compute the time period to the next adjacent position,

l=Vt (4)

where, l is the distance in each step, t is the time period to be calculated. The current at each position is plotted as a function of the time to generate the current pulse signal.

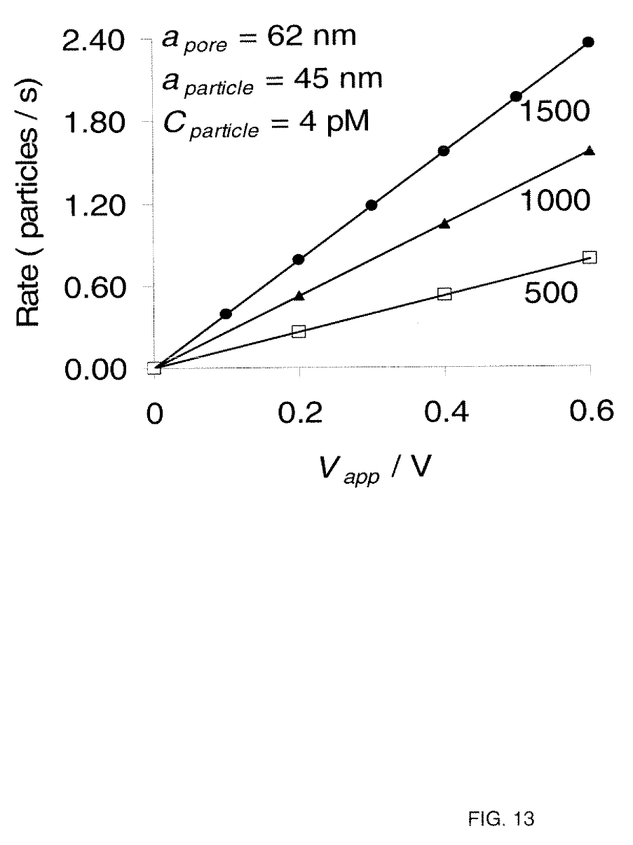

FIG. 13 shows the simulated detection rate as a function of applied voltage. The simulated detection rates are proportional to the applied voltage and the particle charge. These results suggest that the translocation of charged PS nanoparticles is driven by the electrophoretic force (The simulated diffusion rate of nanoparticles is ˜4 orders of magnitude lower than the simulated transfer rate in the presence of a ˜100 mV voltage. Thus, diffusion can be neglected). However, the simulated transfer rate is ˜4× larger than the recorded detection rate at the same conditions (FIG. 6). One possible reason for the discrepancy is that the interactions between the PS spheres and the pore walls are not considered in the simulations. These interactions include the coulomb interaction between the negatively charged particles and the negatively charged glass walls which may slow down the transfer rate of nanoparticles. The simulation does not account for the excess charges in the electrical double layer. As stated before, the ion charges redistribute under the external voltage, causing a decrease in the flux of charged species, including the nanoparticles.

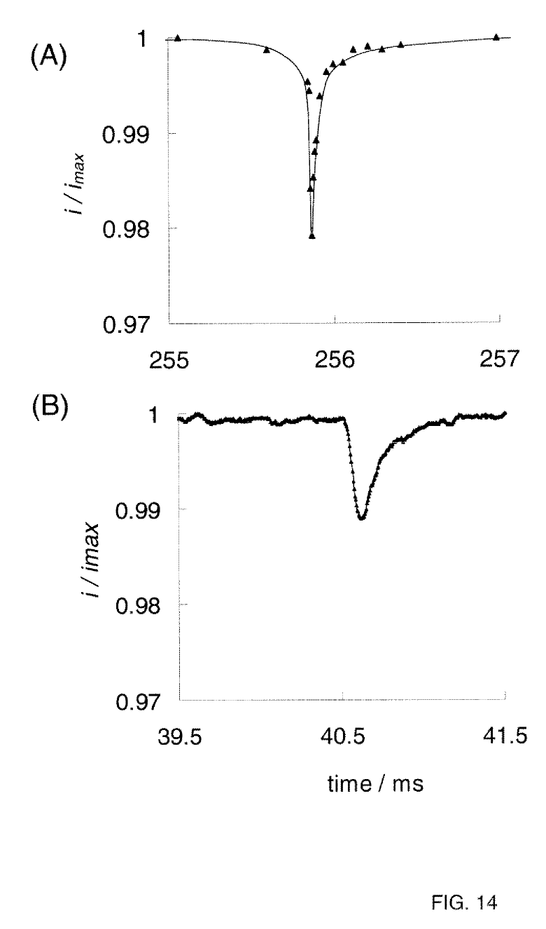

FIG. 14 shows a simulated current pulse (13a) and a typical current pulse recorded in the experiment (13b) for the translocation of 30-nm-radius particle through 64-nm-radius glass nanopore membrane at +0.3 V. The simulated current pulse has a triangle shape, quite similar to the recorded wave. However, the simulated current pulse has a shorter pulse width (˜100 μs) and larger pulse size (Δi/iₘₐₓ=2%), as compared to the recorded pulse (˜200 μs, and Δi/iₘₐₓ=1.2%). Because the interactions and electrical double-layer are not considered in the simulation, the simulated transfer rate of particles is faster than the real transfer rate, which is reflected by the shorter pulse width. The reason for the smaller drop in the i-t trace is that the surface charges on both nanoparticles and the glass pore walls are considered in the simulation. These surface charges bring excess counter ions resulting in an increased electrolyte concentration in the pore as the particle transfers through the pore orifice.

The Statistics of Particle Detection. The translocation of PS nanoparticles through glass nanopore membrane is found to follow a Poisson distribution:

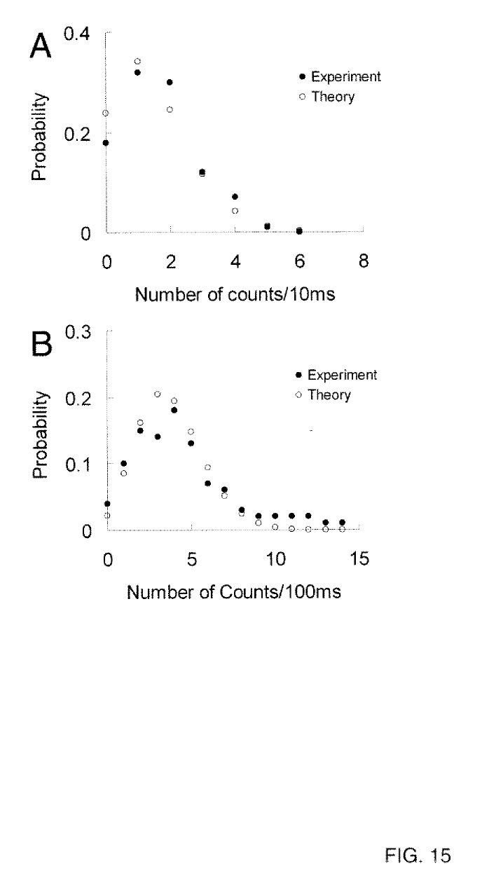

P(k,λΔt)=e^{-λΔt}(λΔt)^{k}/k! (4)

where λ is the average translocation rate (particles/s), Δt is the time interval of counting, k is the number of particles translocated in that time interval, and P is the probability of having k particles translocated in that time interval. FIG. 15(A) shows the probability of observing particle translocations in a 10-ms time interval using 30-nm-radius PS particles. FIG. 15(B) shows the probability of observing particle translocations in a 100-ms interval, using 45-nm-radius PS particles from the data in FIG. 5(A) (1000-1500 pulses of each size particle are counted in the statistics). The good agreement between experiment and the theory shows that the particle translocation is stochastic, and follows a Poisson distribution.

Glass membranes with single conical shaped nanopores have been fabricated and applied to the detection of polystyrene nanoparticles. The conical shape of our glass membrane nanopores has advantages over other conventional membranes that contain cylindrical nanopores, such as short pulse widths and better signal resolutions. Moreover, the glass membrane is easy to fabricate and is portable. In principle, pressure driven flow arising from mechanical forces can be used to drive particles, including neutral particles, across the membrane for analyses analogous to that described in the preceding paragraphs.

A linear dependence is found between the detection rate and the concentration of PS nanoparticles, using particles from as low as sub pM to nM. Particles with lower concentrations can be detected using longer counting times. The translocation of nanoparticles, through conical-shaped glass nanopore membrane is a random process, and obeys a Poisson distribution.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

All references, including publications, patents, and patent applications, cited herein are hereby incorporated by reference to the same extent as if each reference were individually and specifically indicated to be incorporated by reference and were set fort in its entirety herein. The references discussed herein are provided solely for their disclosure prior to the filing date of the present application. Nothing herein is to be construed as an admission that the inventors are not entitled to antedate such disclosure by virtue of prior invention.

What is claimed is:

1. A nanopore device comprising:
a membrane having a thickness, and having a first side and a second side, said first side being opposite to said second side;
a nanopore extending through the membrane, thus forming at least one channel connecting the first and second sides of the membrane, wherein the nanopore has a first opening that opens to the first side of the membrane, and a second opening that opens to the second side of the membrane, and wherein the radius of the first opening of the nanopore ranges from about 2 nm to about 500 nm;
means for applying an electric field between the first and second sides of the membrane;
means for monitoring current flow through the nanopore and/or resistance between the first side and the second side of the membrane; and
means for processing observed current and/or resistance to produce a useful output.

2. The nanopore device of claim 1, wherein the membrane comprises material selected from the group consisting of glass, fused silica, quartz, silicates, and combinations thereof.

3. The nanopore device of claim 2, wherein the nanopore has a conical shape and wherein the first opening of the nanopore is smaller than the second opening of the nanopore.

4. The nanopore device of claim 3, wherein the means for applying an electric field comprises a first electrode and a second electrode.

5. The nanopore device of claim 4, wherein the first electrode is positioned on the first side of the membrane and the second electrode is positioned on the second side of the membrane.

6. The nanopore device of claim 5, wherein the first and/or second electrodes are Ag/AgCl electrodes.

7. The nanopore device of claim 6, wherein the membrane ranges from about 20 μm to 75 μm in thickness.

8. The nanopore device of claim 3, further comprising:
a chamber, wherein the membrane is an integral part of the chamber and wherein the first opening of the nanopore is facing the chamber's exterior and the second opening of the nanopore is facing the chamber's interior;
an electrolyte solution included in the chamber wherein the second opening of the nanopore is immersed in the solution;
a first electrode positioned outside of the chamber; and
a second electrode positioned inside of the chamber wherein at least a portion of the second electrode is immersed in the electrolyte solution.

9. A method of forming a nanopore device, the method comprising:
providing a membrane having a thickness, a first side, and a second side, the first side being opposite to the second side;
providing at least one nanopore extending through the membrane over the thickness of the membrane, thus forming at least one channel connecting the first and second sides of the membrane, wherein the nanopore has a first opening that opens to the membrane's first side, and a second opening that opens to the membrane's second side, and further wherein the first opening of the nanopore ranges from about 2 nm to about 500 nm;
providing means for applying an electric field between the first side and the second side of the membrane;
providing means for monitoring the current flow through the nanopore or resistance between the first side and the second side of the membrane; and
providing means for processing an observed current and/or resistance.

10. A method of counting and analyzing particles, the method comprising:
providing a sample solution containing particles to be analyzed;
contacting the nanopore device of claim 8 with the sample solution such that the first opening of the nanopore is immersed in the sample solution, and the appropriate part of the first electrode is immersed in the sample solution;
applying an appropriate voltage between the first and second electrodes such that the particles from the sample solution are driven to pass across the nanopore;
monitoring the transient change in the electrical resistance, and/or electrical conductivity of the nanopore; and
analyzing the transient change to obtain the concentration, size, shape and/or electrical charge of the particles.

11. The method of claim 10, wherein the particles are selected from the group consisting of cells, bacteria, viruses, polymeric particles, ions, molecules, and mixtures thereof.

12. The method of claim 11, wherein the particles range from about 2 nm to 500 nm.
